(19) United States
(12) Patent Application Publication
(10) Pub. No.: US 2013/0315988 A1
(43) Pub. Date: Nov. 28, 2013

(54) Pharmaceutical Composition

(71) Applicants: Shrinivas Madhukar Purandare, Mumbai (IN); Geena Malhotra, Mumbai (IN)

(72) Inventors: Shrinivas Madhukar Purandare, Mumbai (IN); Geena Malhotra, Mumbai (IN)

(73) Assignee: CIPLA LIMITED, Mumbai (IN)

(21) Appl. No.: 13/822,478

(22) PCT Filed: May 23, 2011
(86) PCT No.: PCT/GB2011/000785

§ 371 Date: May 28, 2013

(30) Foreign Application Priority Data
Sep. 13, 2010 (IN) 2522/MUM/2010
Sep. 14, 2010 (IN) 2539/MUM/2010

(51) Int. Cl. A61K 31/437; A61K 9/16

(52) U.S. Cl. CPC A61K 31/437; A61K 9/16
USPC 424/451; 514/279; 424/489; 424/464; 424/490; 428/402; 427/2.14

(57) ABSTRACT

A composition comprising rifaximin in the form of particles, wherein substantially all the particles have a particle size less than or equal to 2 micrometres.

### CROSS-REFERENCE TO RELATED APPLICATIONS

The present invention claims the benefit of the PCT/GB2011/000785 filed May 23, 2011, which claims priority to Ser. No. 2522/MUM/2010 filed Sep. 13, 2010 and 2539/MUM/2010 filed Sep. 14, 2010.

### FIELD OF INVENTION

The present invention relates to a pharmaceutical composition comprising an antibacterial drug, a process for preparing such pharmaceutical composition, and its use for the treatment and/or prevention of colonic diseases.

### BACKGROUND AND PRIOR ART

One of the major obstacles to the development of highly potent pharmaceutical formulations is the poor water solubility of many drugs. Approximately 40% of potential drugs identified by pharmaceutical companies are poorly soluble in water, which greatly hinders their clinical use. Low water solubility limits the bioavailability and absorption of these agents.

Rifaximin is a semisynthetic antibiotic belonging to the rifamycin class of antimicrobial drugs exhibiting in vitro activity against Gram-positive, Gram-negative and anaerobic bacteria. Rifaximin acts by inhibiting bacterial ribonucleic acid (RNA) synthesis. Rifaximin is chemically named as[(2S,16Z,18E,20S,21S,22R,23R,24R,25S,26S,27S,28E)-5,6,21,23,25-pentahydroxy-27-methoxy-2,4,11,16,20,22,24,26-octamethyl-2,7 (epoxypentadeca-[1,11,13]trienimino)benzofuro [4,5-e]pyrido[1,2-a]-benzimida-zole 1,15(2H)-dione,25-acetate]. Rifaximin has the following chemical formula.

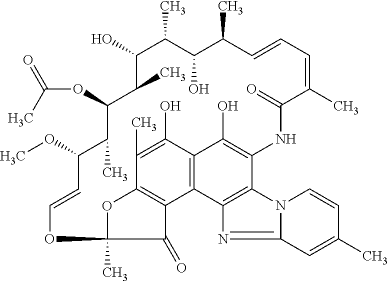

Rifaximin has been indicated for the treatment of traveler's diarrhea caused by non-invasive strains of Escherichia coli, a micro-organism which is not able to penetrate into gastrointestinal (GI) mucosa and therefore remains in contact with gastrointestinal fluids. Rifaximin is highly effective in preventing and treating traveler's diarrhea, with fewer side effects and a low risk of developing antibiotic resistance.

Rifaximin is also approved for the treatment of pathologies whose etiology is in part or totally due to intestinal acute and chronic infections sustained by Gram-positive and Gram-negative bacteria, with diarrhea syndromes, altered intestinal microbial flora, summer diarrhea-like episodes, traveler's diarrhea and enterocolitis, pre- and post-surgery prophylaxis of the infective complications in gastro intestinal surgery; and hyperammonaemia therapy as coadjutant.

Rifaximin is a poorly water-soluble and minimally absorbed (<0.4%) drug with in vitro activity against enteric Gram-negative bacteria including enteric pathogens. [Gerard L et al., Rifaximin, a non-absorbable rifamycin antibiotic for use in nonsystemic gastrointestinal infections. Expert Review of Anti-infective therapy, 3(2), 201-211, (2005)].

It has also been reported that rifaximin is characterized by negligible systemic absorption, due to its chemical and physical characteristics [Descombe J J et al., Pharmacokinetic study of rifaximin after oral administration in healthy volunteers. International Journal of Clinical Pharmacology Research, 14 (2), 51-56, (1994)].

Rifaximin has been described to be endowed with an antibacterial activity similar to the activity of rifampin [Venturini A. P. and Marchi E., Chemiotherapia, 5 (4), 257-256, (1986)]. However, its mechanism of action differs from rifampin in that it is not absorbed through the systemic route after oral administration [Venturini A. P., Chemotherapy, 29, 1-3, (1983) and Cellai L. et al., Chemiotherapia, 3, (6), 373-377, (1984)] due to the zwitterionic nature of the compound, which cannot be absorbed by the gastrointestinal tract [Marchi E. et al., Journal of Medicinal Chemistry., 28, 960-963, (1985)].

Hence in addition to poor water solubility, rifaximin has no systemic absorption which poses a challenge to formulate suitable formulations of rifaximin

Rifaximin is currently available as tablets, granules for oral suspension and ointment, marketed in Europe and U.S.A. and in many other countries. Tablets, for example are currently marketed at the dosage of 200 mg for traveler's diarrhea under the brand name Xifaxan®.

WO2007/047253 discloses methods of increasing the aqueous solubility of an antifungal azole using hydroxybutenyl cyclodextrins.

WO2010/067072 discloses complexes of rifaximin and process for preparing such complexes.

EP0858804 discloses use of oral rifaximin compositions in the treatment of diarrhea from cryptosporidiosis. The rifaximin formulations disclosed in the patent are in the form of tablet, capsule, sugar coated tablet, granules or syrup for oral administration.

U.S. Pat. No. 5,352,679 discloses use of rifaximin in formulations for treatment of gastric dyspepsia caused by Helicobacter pylori bacteria. The rifaximin formulations disclosed in the patent are in the form of tablet, capsule, sugar coated tablet, granules or syrup for oral administration.

Several strategies and formulations have been employed to overcome these limitations of solubility and poor systemic absorption. Although existing strategies such as complexing drugs with cyclodextrins, conjugation to dendrimers, salt formation of ionizable drugs and the use of co-solvents have been shown to improve drug solubility, solubilization methods that can improve the absorption of the drug are still highly desirable.

Hence, there still exists a need to develop and formulate a suitable composition of rifaximin that overcome the problems mentioned in the prior art.

### OBJECT OF THE INVENTION

The object of the present invention is to provide a pharmaceutical composition of nanosized rifaximin having improved surface area and solubility.

Another object of the present invention is to provide a process for preparing the pharmaceutical composition comprising nanosized rifaximin

Yet another object of the present invention is to provide a method for treatment and/or prevention of colonic diseases which comprises administering a pharmaceutical composition comprising nanosized rifaximin

### SUMMARY OF THE INVENTION

According to one aspect of the present invention there is provided a pharmaceutical composition comprising rifaximin or a pharmaceutically acceptable salt, solvate, derivative, hydrate, enantiomer, polymorph, prodrug, complex, or mixture thereof.

According to another aspect of the present invention there is provided a pharmaceutical composition comprising rifaximin or a pharmaceutically acceptable salt, solvate, derivative, hydrate, enantiomer, polymorph, prodrug, complex or mixture thereof wherein the rifaximin is in the nanosize range.

According to yet another aspect of the present invention there is provided a process for preparing a pharmaceutical composition comprising rifaximin or a pharmaceutically acceptable salt, solvate, derivative, hydrate, enantiomer, polymorph, prodrug, complex or mixture thereof wherein the rifaximin is in the nanosize range.

According to a further aspect of the present invention there is provided a method of treatment and/or prevention of colonic diseases using a pharmaceutical composition comprising rifaximin or a pharmaceutically acceptable salt, solvate, derivative, hydrate, enantiomer, polymorph, prodrug, complex or mixture thereof wherein the rifaximin is in the nanosize range.

### BRIEF DESCRIPTION OF THE FIGURES

FIG. 1. Graph showing dissolution data.

### DETAILED DESCRIPTION OF THE INVENTION

Rifaximin is a poorly water-soluble drug exhibiting minimal absorption. Rifaximin is also characterized by negligible systemic absorption, due to its chemical and physical characteristics and hence it has been difficult to formulate rifaximin in any suitable dosage form

The inventors of the present invention have found that the solubility properties of rifaximin were improved by nanosizing rifaximin thus leading to better localized therapeutic effect for example in the colon.

Nanonization of hydrophobic or poorly water-soluble drugs generally involves the production of drug nanocrystals through either chemical precipitation (bottom-up technology) or disintegration (top-down technology). Different methods may be utilized to reduce the particle size of the hydrophobic or poorly water soluble drugs. [Huabing Chen et al., discusses the various methods to develop nanoformulations in “Nanonization strategies for poorly water-soluble drugs,” Drug Discovery Today, Volume 00, Number 00, March 2010].

Nanosizing leads to increase in the exposure of surface area of rifaximin particles leading to an increase in the rate of dissolution.

The present invention thus provides a pharmaceutical composition comprising rifaximin wherein rifaximin is in the nanosize range.

The term “rifaximin” is used in broad sense to include not only “rifaximin” per se but also its pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs, pharmaceutically acceptable prodrugs, pharmaceutically acceptable complexes etc.

The nanoparticles of the present invention can be obtained by any of the process such as but not limited to milling, precipitation and homogenization.

The pharmaceutical composition of the present invention comprises rifaximin having an effective particle size range of less than 2000 nm, preferably below 1000 nm.

According to one embodiment of the present invention, the process of milling comprises dispersing rifaximin particles in a liquid dispersion medium in which rifaximin is poorly soluble, followed by applying mechanical means in the presence of grinding media to reduce the particle size of rifaximin to the desired effective average particle size.

According to another embodiment of the present invention, the process of precipitation involves the formation of crystalline or semi-crystalline rifaximin nanoparticles by nucleation and the growth of drug crystals. In a typical procedure, drug molecules are first dissolved in an appropriate organic solvent such as acetone, tetrahydrofuran or N-methyl-2-pyrrolidone at a super saturation concentration to allow for the nucleation of drug seeds. Drug nanocrystals are then formed by adding the organic mixture to an antisolvent like water in the presence of stabilizers such surfactants. The choice of solvents and stabilizers and the mixing process are key factors to control the size and stability of the drug nanocrystals.

According to one another embodiment of the present invention, the process of homogenization involves passing a suspension of crystalline rifaximin and stabilizers through the narrow gap of a homogenizer at high pressure (500-2000 bar). The pressure creates powerful disruptive forces such as cavitation, collision and shearing, which disintegrate coarse particles to nanoparticles.

According to yet another embodiment of the present invention, the process of high pressure homogenization comprises rifaximin presuspension (containing rifaximin in the micrometer range) by subjecting the rifaximin to air jet milling in the presence of an aqueous surfactant solution. The presuspension is then subjected to high-pressure homogenization in which it passes through a very small homogenizer gap of ˜25 nm which leads to a high streaming velocity. High-pressure homogenization is based on the principle of cavitations (i.e., the formation, growth, and implosive collapse of vapor bubbles in a liquid).

According to one more embodiment of the present invention, the process of spray-freeze drying involves the atomization of an aqueous rifaximin solution into a spray chamber filled with a cryogenic liquid (liquid nitrogen) or halocarbon refrigerant such as chlorofluorocarbon or fluorocarbon. The water is removed by sublimation after the liquid droplets solidify.

According to a still another embodiment of the present invention, the process of supercritical fluid technology involves controlled crystallization of rifaximin from dispersion in supercritical fluids, carbon dioxide.

According to another embodiment of the present invention, the process of double emulsion/solvent evaporation technique involves preparation of oil/water (o/w) emulsions with subsequent removal of the oil phase through evaporation. The emulsions are prepared by emulsifying the organic phase containing rifaximin, polymer and organic solvent in an aqueous solution containing emulsifier. The organic solvent diffuses out of the polymer phase and into the aqueous phase, and is then evaporated, forming rifaximin-loaded polymeric nanoparticles.

According to a further embodiment of the present invention, the process of PRINT (Particle replication in non-wetting templates) involves utilization of a low surface energy fluoropolymeric mold that enables high-resolution imprint lithography, to fabricate a variety of organic particles. PRINT can precisely manipulate particle size of rifaximin ranging from 20 nm to more than 100 nm.

According to one further embodiment of the present invention, the process of thermal condensation involves use of capillary aerosol generator (CAG) to produce high concentration condensation submicron to micron sized aerosols from rifaximin solutions.

According to still further embodiment of the present invention, the process of ultrasonication involves application of ultrasound during particle synthesis or precipitation, which leads to smaller particles of rifaximin and increased size uniformity.

According to another embodiment of the present invention, the process of spray drying involves supplying the feed solution at room temperature and pumping it through the nozzle where it is atomized by the nozzle gas. The atomized solution is then dried by preheated drying gas in a special chamber to remove water moisture from the system, thus forming dry particles of rifaximin

According to a preferred embodiment of the present invention, the namomilled rifixamin may be obtained by nanomilling of rifaximin with at least one surface stabilizer, at least one viscosity building agent and at least one polymer.

The present invention thus provides a pharmaceutical composition comprising granules of rifaximin, wherein rifaximin is in the nanosize range and the granules comprise at least one surface stabilizer, at least one viscosity building agent and at least one polymer along with rifaximin and optionally other pharmaceutically acceptable carriers.

Surface stabilizer, according to the present inventions, means surfactants that are capable of stabilizing the increased surfaced charge of the nanomilled drug. Suitable amphoteric, non-ionic, cationic or anionic surfactants may be included in the pharmaceutical composition of the present invention.

According to the present invention, surfactant may comprise one or more, but not limited to Polysorbates, Sodium dodecyl sulfate (sodium lauryl sulfate), Lauryl dimethyl amine oxide, Docusate sodium, Cetyl trimethyl ammonium bromide (CTAB) Polyethoxylated alcohols, Polyoxyethylene sorbitan, Octoxynol, N,N-dimethyldodecylamine-N-oxide, Hexadecyltrimethylammonium bromide, Polyoxyl 10 lauryl ether, Brij, Bile salts (sodium deoxycholate, sodium cholate), Polyoxyl castor oil, Nonylphenol ethoxylate, Cyclodextrins, Lecithin, Methylbenzethonium chloride. Carboxylates, Sulphonates, Petroleum sulphonates, alkylbenzenesulphonates, Naphthalenesulphonates, Olefin sulphonates, Alkyl sulphates, Sulphates, Sulphated natural oils & fats, Sulphated esters, Sulphated alkanolamides, Alkylphenols, ethoxylated & sulphated, Ethoxylated aliphatic alcohol, polyoxyethylene surfactants, carboxylic esters Polyethylene glycol esters, Anhydrosorbitol ester & it's ethoxylated derivatives, Glycol esters of fatty acids, Carboxylic amides, Monoalkanolamine condensates, Polyoxyethylene fatty acid amides, Quaternary ammonium salts, Amines with amide linkages, Polyoxyethylene alkyl & alicyclic amines, N,N,N,N tetrakis substituted ethylenediamines 2-alkyl 1-hydroxyethyl 2-imidazolines, N-coco 3-aminopropionic acid/sodium salt, N-tallow 3-iminodipropionate disodium salt, N-carboxymethyl n dimethyl n-9 octadecenyl ammonium hydroxide, n-cocoamidethyl n-hydroxyethylglycine sodium salt or mixtures thereof etc.

Viscosity builders means, excipients that are capable of stabilizing the nanoparticles by increasing the viscosity of the formulation and thus preventing physical interaction of nanoparticles under the operating conditions employed.

According to the present invention, viscosity builders, may comprise one or more, but not limited to derivatives of sugars, such as lactose, saccharose, hydrolyzed starch (maltodextrin) etc or mixtures thereof.

Polymers or polymers blends, according to the present invention, may comprise one or more hydrophilic polymers, but not limited to cellulose derivates like hydroxypropylcellulose, hydroxymethylcellulose, hydroxypropylmethylcellulose, methylcellulose polymers hydroxyethylcellulose, sodium carboxymethylcellulose, carboxymethylene and carboxymethyl hydroxyethylcellulose; acrylics like acrylic acid, acrylamide, and maleic anhydride polymers, acacia, gum tragacanth, locust bean gum, guar gum, or karaya gum, agar, pectin, carrageenan, gelatin, casein, zein and alginates, carboxypolymethylene, bentonite, magnesium aluminum silicate, polysaccharides, modified starch derivatives and copolymers or mixtures thereof.

In one embodiment the percentage weight of active ingredient in the slurry ranges from 5% to 60% w/w.

The nanoparticulate rifaximin compositions of the invention can be administered to a subject via any conventional means including, but not limited to, orally, rectally, ocularly, parenterally (e.g., intravenous, intramuscular, or subcutaneous), intracisternally, intravaginally, intraperitoneally, locally (e.g., powders, ointments or drops), or buccal route.

In an embodiment, the rifaximin composition according to the invention is not a rectal formulation.

In an embodiment, the rifaximin composition according to the invention is not a rectal foam formulation.

Moreover, the nanoparticulate rifaximin compositions of the invention can be formulated into any suitable dosage form, including but not limited to liquid dispersions, gels, aerosols, ointments, creams, controlled release formulations, lyophilized formulations, tablets, capsules, delayed release formulations, extended release formulations, pulsatile release formulations, and mixed immediate release and controlled release formulations.

The nanomilled rifaximin compositions can be formulated for parenteral injection (e.g., intravenous, intramuscular, or subcutaneous), oral administration in solid, liquid, or aerosol form, foams (vaginal, rectal), vaginal, rectal, ocular, local (powders, ointments or drops), buccal, intracistemal, intraperitoneal, or topical administration, and the like.

Nanoparticulate rifaximin compositions suitable for parenteral injection may comprise physiologically acceptable sterile aqueous or nonaqueous solutions, dispersions, suspensions or emulsions, and sterile powders for reconstitution into sterile injectable solutions or dispersions.

Examples of suitable aqueous and nonaqueous carriers, diluents, solvents, or vehicles include but are not limited to, water, ethanol, polyols (propyleneglycol, polyethyleneglycol, glycerol, and the like) and suitable mixtures thereof, vegetable oils and injectable organic esters.

The nanoparticulate rifaximin compositions may also contain excipients such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like.

Solid dosage forms for oral administration include, but are not limited to, capsules, tablets, pills, powders, and granules. In such solid dosage forms, the active agent is admixed with at least one of the following: (a) one or more inert excipients (or carriers) (b) fillers or extenders (c) binders (d) humectants (e) disintegrating agents (f) solution retarders (g) absorption accelerators (h) wetting agents (i) adsorbents and (j) lubricants. For capsules, tablets, and pills, the dosage forms may also comprise buffering agents.

The nanomilled granules obtained, according to the present invention, may either be encapsulated in capsules or be compressed to form tablets or may be provided as sachets or be provided as powders for reconstitution.

The solid dosage form, according to the present invention, may also optionally be coated. More preferably, the formulation may be seal coated and further enteric coated.

According to an embodiment of the present invention, there is a seal coat between the core containing rifaximin, and the enteric coat. The seal coat comprises one or more pharmaceutically acceptable film-forming polymers and pharmaceutically acceptable excipients(s). The seal coat provides a smooth base for the application of the enteric coat, prolongs the resistance of the core to the acidic conditions, improves stability by minimizing the interaction between drug in the core and the enteric polymer in the enteric layer from coming into direct contact with each other; and also improves stability of drug from light exposure. The smoothing function of the separating coat is purely mechanical, the objective of which is to improve the coverage of the enteric coat and to avoid thin spots in it, caused by bumps and irregularities on the core.

According to the present invention, the seal coat comprises film forming polymeric materials, such as but not limited to, hydroxypropylmethylcellulose, hydroxypropylcellulose, polyvinylpyrrolidone, methylcellulose, carboxymethylcellulose, hypromellose, acacia, gelatin to increase adherence and coherence of the seal coat.

According to another embodiment of the present invention, the enteric coat is present over the seal coat. The enteric coat comprises of materials such as, but not limited to, neutralized methacrylic acid copolymers such as, EUDRAGIT L 30 D-55, EUDRAGIT L100-55, EUDRAGIT S 100, EASTACRYL 30D, KOLLICOAT MAE 30 DP, KOLLICOAT MAE 100 P; cellulose acetate phthalate, hydroxypropyl methylcellulose phthalate and combinations thereof.

According to the present invention, the enteric coat of the present invention may include a plasticizer, surfactant, pigments, anti-adherents, opacifying agents, colorants and the like, which are routinely employed in the preparation of coating solution or suspension.

The plasticizers used in the present invention may comprise polyethylene glycol, tributyl sebacate, acetylated monoglycerides, glycerin, triacetin, phthalate esters, castor oil, sorbitol, polysorbates such as sorbitan monolaurate (Span 20), sorbitan monopalmitate, sorbitan monostearate, sorbitan monoisostearate; citrate ester type plasticizers like triethyl citrate, citrate phthalate; propylene glycol, glycerin, polyethylene glycol (low & high molecular weight), dibutyl sebacate, tributyl sebacate; dibutyltartrate, dibutyl phthalate, glycerol palmitosterate and mixtures thereof.

The anti-adherent used in the present invention may comprise talc, magnesium stearate, fumed silica, micronized silica and silicon dioxide and mixtures thereof.

Alternatively, the nanomilled slurry may also be used to formulate liquid dosage forms.

Liquid nanoparticulate rifaximin dosage forms for oral administration include pharmaceutically acceptable emulsions, solutions, suspensions, syrups, and elixirs. In addition to rifaximin, the liquid dosage forms may comprise inert diluents commonly used in the art, such as water or other solvents, solubilizing agents, and emulsifiers.

Besides such inert diluents, the composition can also include adjuvants, such as wetting agents, emulsifying and suspending agents, sweetening, flavoring, and perfuming agents.

In addition to rifaximin, the liquid dosage forms may comprise inert diluents, such as water or other solvents, solubilizing agents, suspending agents, emulsifying agents, sweetening agents, flavoring agents, perfuming agents, pH adjusting agents and preservatives.

Suitable excipients may be used for formulating the various dosage forms according to the present invention.

Emulsifying agents may comprise one or more, but not limited to, ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures thereof.

Suspending agents may comprise one or more, but not limited to, methylcellulose, hydroxypropylmethylcellulose, microcrystalline cellulose, hydroxybutylmethylcellulose hydroxyethylmethylcellulose, ethylhydroxyethylcellulose, hydroxyethylcellulose, hydroxypropylcellulose, carboxymethylcellulose sodium, xanthan gum, silicon dioxide and mixtures thereof.

According to the present invention, pharmaceutically acceptable carriers, diluents or fillers for use in the pharmaceutical composition of the present invention may comprise one or more, but not limited to lactose (for example, spray-dried lactose, α-lactose, β-lactose) lactose available under the trade mark Tablettose, various grades of lactose available under the trade mark Pharmatose or other commercially available forms of lactose, lactitol, saccharose, sorbitol, mannitol, dextrates, dextrins, dextrose, maltodextrin, croscarmellose sodium, microcrystalline cellulose (for example, microcrystalline cellulose available under the trade mark Avicel), hydroxypropylcellulose, L-hydroxypropylcellulose (low substituted), hydroxypropyl methylcellulose (HPMC), methylcellulose polymers (such as, for example, Methocel A, Methocel A4C, Methocel A15C, Methocel A4M), hydroxyethylcellulose, sodium carboxymethylcellulose, carboxymethylene, carboxymethyl hydroxyethylcellulose and other cellulose derivatives, starches or modified starches (including potato starch, corn starch, maize starch and rice starch) and mixtures thereof.

According to the present invention, glidants and lubricants may also be incorporated in the pharmaceutical composition of the present invention, which may comprise one or more, but not limited to stearic acid and pharmaceutically acceptable salts or esters thereof (for example, magnesium stearate, calcium stearate, sodium stearyl fumarate or other metallic stearate), talc, waxes (for example, microcrystalline waxes) and glycerides, light mineral oil, PEG, silica acid or a derivative or salt thereof (for example, silicates, silicon dioxide, colloidal silicon dioxide and polymers thereof, crospovidone, magnesium aluminosilicate and/or magnesium alumino metasilicate), sucrose ester of fatty acids, hydrogenated vegetable oils (for example, hydrogenated castor oil), or mixtures thereof.

According to the present invention, suitable binders may also be present in the in the pharmaceutical composition of the present invention, which may comprise one or more, but not limited to polyvinyl pyrrolidone (also known as povidone), polyethylene glycol(s), acacia, alginic acid, agar, calcium carragenan, cellulose derivatives such as ethyl cellulose, methyl cellulose, hydroxypropyl cellulose, hydroxypropyl methyl cellulose, sodium carboxymethylcellulose, dextrin, gelatin, gum arabic, guar gum, tragacanth, sodium alginate, or mixtures thereof or any other suitable binder.

According to the present invention, suitable disintegrants may also be present in the in the pharmaceutical composition of the present invention, which may comprise one or more, but not limited to hydroxylpropyl cellulose (HPC), low density HPC, carboxymethylcellulose (CMC), sodium CMC, calcium CMC, croscarmellose sodium; starches exemplified under examples of fillers and also carboxymethyl starch, hydroxylpropyl starch, modified starch; crystalline cellulose, sodium starch glycolate; alginic acid or a salt thereof, such as sodium alginate or their equivalents and mixtures thereof.

Further, the pharmaceutical composition according to the present invention may further comprise at least one additional active ingredient.

Additional active agents may be selected from, but not limited to one or more of anti-inflammatory agents, steroids (e.g. corticosteroids), additional antibiotics, anti-fungal agents, anti-viral agents, analgesics, or anti-neoplastic agents.

Suitable antibiotics include, but are not limited to, dapsone, chloramphenicol, neomycin, cefaclor, cefadroxil, cephalexin, cephradine, erythromycin, clindamycin, lincomycin, amoxicillin, ampicillin, bacampicillin, carbenicillin, did oxacillin, cyclacillin, picloxacillin, hetacillin, methicillin, nafcillin, penicillin, polymyxin, tetracycline, amphotericin-b, candicidin, dermostatin, filipin, fungichromin, hachimycin, hamycin, lucensomycin, mepartricin, natamycin, nystatin, pecilocin, perimycin, azaserine, griseofulvin, oligomycins, neomycin undecylenate, pyrroinitrin, siccanin, tubercidin, viridin, picloxacillin, hetacillin, methicillin, nafcillin, penicillin, polymyxin or tetracycline.

Suitable anitfungal agents include but are not limited to, allylamines such as butenafine, naftifine, imidazoles such as bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole, triazoles such as fluconazole, itraconazole, saperconazole, terconazole, and others such as acrisorcin, amorolfine, biphenamine, bromosalicylchloranilide, buclosamide, calcium propionate, chlophenesin, ciclopirox, cloxyquin, coparaffinate, diamthazole, dihydrochloride, exalamide, flucytosine, halethazole, hexetidine, Ioflucarban, nifuratel, potassium iodide, propionates, propionic acid, pyrithione, salicylanilide, sulbentine, tenonitrozole, triacetin, ujothion, undecylenic acid.

Antifungal agents may also include, polyenes such as amphotericin-b, candicidin, dermostatin, filipin, fungichromin, hachimycin, hamycin, lucensomycin, mepartricin, natamycin, nystatin, pecilocin, perimycin, azaserine, griseofulvin, oligomycins, neomycin undecylenate, pyrroinitrin, siccanin, tubercidin, viridin, allylamines such as butenafine, naftifine, imidazoles such as bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole, triazoles such as fluconazole, itraconazole, saperconazole, terconazole, acrisorcin, amorolfine, biphenamine, bromosalicylchloranilide, buclosamide, calcium propionate, chlophenesin, ciclopirox, cloxyquin, coparaffinate, diamthazole, dihydrochloride, exalamide, flucytosine, halethazole, hexetidine, Ioflucarban, nifuratel, potassium iodide, propionates, propionic acid, pyrithione, salicylanilide, sulbentine, tenonitrozole, triacetin, ujothion or undecylenic acid.

Other therapeutic agents can include a steroidal or non-steroidal antiinflammatory agent. Non-steroidal anti-inflammatory agents, include, but are not limited to, aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicarn, isoxicam; salicylic acid derivatives, including aspirin, sodium salicylate, choline magnesium trisalicylate, salsalate, diflunisal, salicylsalicylic acid, sulfasalazine, and olsalazin; para-aminophennol derivatives including acetaminophen and phenacetin; indole and indene acetic acids, including indomethacin, sulindac, and etodolac; heteroaryl acetic acids, including tolmetin, diclofenac, and ketorolac; anthranilic acids (fenamates), including mefenamic acid, and meclofenamic acid; enolic acids, including oxicams (piroxicam, tenoxicam), and pyrazolidinediones (phenylbutazone, oxyphenthartazone); and alkanones, including nabumetone and pharmaceutically acceptable salts thereof and mixtures thereof.

Suitable corticosteroids include but are not limited to, hydrocortisone, i.e., 11-17-21-trihydroxypregn-4-ene-3,20-dione or Cortisol, Cortisol acetate, hydrocortisone phosphate, hydrocortisone 21-sodium succinate, hydrocortisone tebutate, corticosterone, corticosterone acetate, cortisone, cortisone acetate, cortisone 21B-cyclopentanepropionate, cortisone phosphate, triamcinolone hexacetonide, dexamethasone phosphate, desonide, betamethasone dipropionate, mometasone furate.

Antineoplastic agents may also be included in the pharmaceutical composition of the present invention along with rifaximin which include, but are not limited to, vincristine, vinblastine, vindesine, busulfan, chlorambucil, spiroplatin, cisplatin, carboplatin, methotrexate, adriamycin, mitomycin, bleomycin, cytosine arabinoside, arabinosyl adenine, mercaptopurine, mitotane, procarbazine, dactinomycin (antinomycin D), daunorubicin, doxorubicin hydrochloride, taxol, plicamycin, aminoglutethimide, estramustine, flutamide, leuprolide, megestrol acetate, tamoxifen, testolactone, trilostane, amsacrine (m-AMSA), asparaginase (L-asparaginase), etoposide, and interferon a-2a and 2b.

Antiviral agents may also be included in the pharmaceutical composition of the present invention along with the rifaximin which include, but are not limited to, acyclovir, amantadine, azidothymidine, ribavirin and vidarabine.

In a further embodiment, the pharmaceutical composition according to the present invention may be used to treat a bacterial infection, e.g., acute hemorrhoidal disease, irritable bowel syndrome, travelers' diarrhea, small intestinal anal disease, Crohn's disease, chronic pancreatitis, pancreatic insufficiency, colitis, hepatic encephalopathy, antibiotic associated colitis, and/or diverticular disease.

The present invention further provides a method of treatment and/or prevention of colonic diseases, which method comprising administering to a subject in need thereof an effective amount of rifaximin, wherein the rifaximin is in the nanosize range.

There is further provided a process for preparing the pharmaceutical compositions which process comprises

(a) Homogenizing the dispersion of rifaximin, docusate sodium, sucrose, HPMC (b) Nanomilling the homogenized dispersion obtained in step (a)

(b) Adsorbing the nanomilled slurry obtained in step (b) on a mixture of lactose monohydrate, microcrystalline cellulose and crospovidone to form granules.

According to one embodiment the pharmaceutical composition of the present invention, may be prepared by a process which comprises (a) preparing a dispersion of rifaximin with Docusate sodium, HPMC, sodium lauryl sulphate and sucrose in purified water under stirring conditions (b) homogenizing the dispersion of step (a) and then nanomilling the homogenized dispersion (c) adsorbing the nanomilled drug by spraying the nanomilled slurry on lactose monohydrate, microcrystalline cellulose and crospovidone mixture in fluidized bed granulator (d) drying and blending the granules obtained (e) lubricating the granules and finally compressing into tablets (f) the tablets obtained were seal coated and then enteric coated.

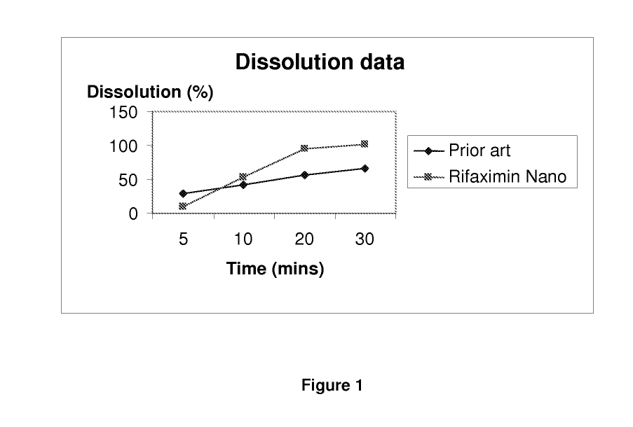

The nanomilled rifaximin composition prepared according to the present invention exhibited a dissolution profile which is showing an improvement over the prior art composition as evident from FIG. 1. This might further lead to a considerably enhanced bioavailability of the active ingredient compared to that obtained with the compositions of the prior art.

The following examples are for the purpose of illustration of the invention only and is not intended in any way to limit the scope of the present invention.

### Example 1

| Sr. No. | Ingredients | Qty mg/tablet |
|---|---|---|
| | Binder Solution | |
| 1. | Rifaximin | 200.00 |
| 2. | Docusate Sodium IP | 2.00 |
| 3. | Hydroxypropylmethylcellulose 3 cps IP | 40.00 |
| 4. | Sodium lauryl sulphate IP | 5.50 |
| 5. | Sucrose IP | 69.00 |
| 6. | Purified water IP | q.s |
| | Dry Mix | |
| 7. | Lactose Monohydrate(200 mesh) IP | 200.00 |
| 8. | Microcrystalline Cellulose IP (Avicel PH 101) | 200.00 |
| 9. | Crospovidone IP | 25.00 |
| | Lubrication | |
| 10. | Crospovidone IP | 30.00 |
| 11. | Magnesium Stearate IP | 03.50 |
| | Total | 775.00 |
| | Seal Coating | |
| 12. | Hydroxypropylmethylcellulose 3 cps IP | 15.00 |
| 13. | Isopropyl Alcohol IP | q.s |
| 14. | Dichloromethane BP | q.s |
| | Total | 790.00 |
| | Enteric Coating | |
| 15. | Eudragit L (30% dispersion) | 26.00 |
| 16. | Talc | 13.00 |
| 17. | Tri-Ethyl Citrate | 13.00 |
| 18. | Water | q.s. |
| | Total | 842.00 |

Process:

1. Dispersion of rifaximin with Docusate sodium, HPMC, sodium lauryl sulphate and sucrose was prepared in purified water under stirring conditions

2. Above dispersion was homogenized and then Nanomilled

3. Nanomilled drug slurry was adsorbed by spraying on lactose monohydrate, microcrystalline cellulose and crospovidone mixture in a fluidized bed granulator.

4. Granules obtained were sized and lubricated.

5. Lubricated granules were finally compressed into tablets

6. The tablets obtained were seal coated and then enteric coated.

### Example 2

| Sr. No. | Ingredients | Qty mg/tablet |
|---|---|---|
| | Binder Solution | |
| 1. | Rifaximin | 200.00 |
| 2. | Docusate Sodium IP | 2.00 |
| 3. | Hydroxypropylmethylcellulose 3 cps IP | 40.00 |
| 4. | Sodium lauryl sulphate IP | 5.50 |
| 5. | Sucrose IP | 69.00 |
| 6. | Purified water IP | q.s |
| | Dry Mix | |
| 7. | Lactose Monohydrate(200 mesh) IP | 200.00 |
| 8. | Microcrystalline Cellulose IP (Avicel PH 101) | 200.00 |
| 9. | Crospovidone IP | 25.00 |
| | Lubrication | |
| 10. | Crospovidone IP | 30.00 |
| 11. | Magnesium Stearate IP | 03.50 |
| | Total | 775.00 |
| | Seal Coating | |
| 12. | Hydroxypropylmethylcellulose 3 cps IP | 15.00 |
| 13. | Isopropyl Alcohol IP | q.s |
| 14. | Dichloromethane BP | q.s |
| | Total | 790.00 |
| | Enteric Coating | |
| 15. | Hydroxypropyl Methylcellulose Pthalate | 26.00 |
| 16. | Triacetin | 13.00 |
| 17. | Isopropyl Alcohol IP | q.s. |
| 18. | Dichloromethane BP | q.s. |
| | Total | 818.60 |

Process:

1. Dispersion of rifaximin with Docusate sodium, HPMC, sodium lauryl sulphate and sucrose was prepared in purified water under stirring conditions

2. Above dispersion was homogenized and then Nanomilled

3. Nanomilled drug slurry was adsorbed by spraying on lactose monohydrate, microcrystalline cellulose and crospovidone mixture in a fluidized bed granulator.

4. Granules obtained were sized and lubricated.

5. Lubricated granules were finally compressed into tablets

6. The tablets obtained were seal coated and then enteric coated.

### Example 3

| Sr. No. | Ingredients | Qty mg/tablet |
|---|---|---|
| | Binder Solution | |
| 1. | Rifaximin | 200.00 |
| 2. | Docusate Sodium IP | 2.00 |
| 3. | Hydroxypropylmethylcellulose 3 cps IP | 40.00 |
| 4. | Sodium lauryl sulphate IP | 5.50 |
| 5. | Sucrose IP | 69.00 |
| 6. | Purified water IP | q.s |
| | Dry Mix | |
| 7. | Lactose Monohydrate(200 mesh) IP | 200.00 |
| 8. | Microcrystalline Cellulose IP (Avicel PH 101) | 200.00 |
| 9. | Crospovidone IP | 25.00 |
| | Lubrication | |
| 10. | Crospovidone IP | 30.00 |
| 11. | Magnesium Stearate IP | 03.50 |
| | Total | 775.00 |
| | Seal Coating | |
| 12. | Hydroxypropylmethylcellulose 3 cps IP | 15.00 |
| 13. | Isopropyl Alcohol IP | q.s |
| 14. | Dichloromethane BP | q.s |
| | Total | 790.00 |
| | Enteric Coating | |
| 15. | Cellulose Acetate Pthalate | 26.00 |
| 16. | Triacetin | 2.60 |
| 17. | Isopropyl Alcohol IP | q.s. |
| 18. | Dichloromethane BP | q.s. |
| | Total | 818.60 |

Process:

1. Dispersion of rifaximin with Docusate sodium, HPMC, sodium lauryl sulphate and sucrose was prepared in purified water under stirring conditions

2. Above dispersion was homogenized and then Nanomilled

3. Nanomilled drug slurry was adsorbed by spraying on lactose monohydrate, microcrystalline cellulose and crospovidone mixture in a fluidized bed granulator.

4. Granules obtained were sized and lubricated.

5. Lubricated granules were finally compressed into tablets

6. The tablets obtained were seal coated and then enteric coated.

### Example 4

| Sr. No. | Ingredients | Qty mg/tablet |
|---|---|---|
| | Binder Solution | |
| 1. | Rifaximin | 200.00 |
| 2. | Docusate Sodium IP | 2.00 |
| 3. | Hydroxypropylmethylcellulose 3 cps IP | 40.00 |
| 4. | Sodium lauryl sulphate IP | 5.50 |
| 5. | Sucrose IP | 69.00 |
| 6. | Purified water IP | q.s |
| | Dry Mix | |
| 7. | Lactose Monohydrate(200 mesh) IP | 200.00 |
| 8. | Microcrystalline Cellulose IP (Avicel PH 101) | 200.00 |
| 9. | Crospovidone IP | 25.00 |
| | Lubrication | |
| 10. | Crospovidone IP | 30.00 |
| 11. | Magnesium Stearate IP | 03.50 |
| | Total | 775.00 |
| | Seal Coating | |
| 12. | Hydroxypropylmethylcellulose 3 cps IP | 15.00 |
| 13. | Isopropyl Alcohol IP | q.s |
| 14. | Dichloromethane BP | q.s |
| | Total | 790.00 |
| | Enteric Coating | |
| 15. | Eudragit S100 | 26.00 |
| 16. | Talc | 13.00 |
| 17. | Tri-Ethyl Citrate | 13.00 |
| 18. | Water | q.s. |
| | Total | 842.00 |

Process:

1. Dispersion of rifaximin with Docusate sodium, HPMC, sodium lauryl sulphate and sucrose was prepared in purified water under stirring conditions

2. Above dispersion was homogenized and then Nanomilled

3. Nanomilled drug slurry was adsorbed by spraying on lactose monohydrate, microcrystalline cellulose and crospovidone mixture in a fluidized bed granulator.

4. Granules obtained were sized and lubricated.

5. Lubricated granules were finally compressed into tablets

6. The tablets obtained were seal coated and then enteric coated.

### Example 5

| Sr. No. | Ingredients | Qty mg/tablet |
|---|---|---|
| | Binder Solution | |
| 1. | Rifaximin | 200.00 |
| 2. | Docusate Sodium IP | 2.00 |
| 3. | Hydroxypropylmethylcellulose 3 cps IP | 40.00 |
| 4. | Sodium lauryl sulphate IP | 5.50 |
| 5. | Sucrose IP | 69.00 |
| 6. | Purified water IP | q.s |
| | Dry Mix | |
| 7. | Lactose Monohydrate(200 mesh) IP | 200.00 |
| 8. | Microcrystalline Cellulose IP (Avicel PH 101) | 200.00 |
| 9. | Crospovidone IP | 25.00 |
| | Lubrication | |
| 10. | Crospovidone IP | 30.00 |
| 11. | Magnesium Stearate IP | 03.50 |
| | Total | 775.00 |
| | Seal Coating | |
| 12. | Hydroxypropylmethylcellulose 3 cps IP | 15.00 |
| 13. | Isopropyl Alcohol IP | q.s |
| 14. | Dichloromethane BP | q.s |
| | Total | 790.00 |
| | Enteric Coating | |
| 15. | Eudragit L (30% dispersion) | 13.00 |
| 16. | Eudragit S100 | 13.00 |
| 17. | Talc | 13.00 |
| 18. | Tri-Ethyl Citrate | 13.00 |
| 19. | Water | q.s. |
| | Total | 842.00 |

Process:

1. Dispersion of rifaximin with Docusate sodium, HPMC, sodium lauryl sulphate and sucrose was prepared in purified water under stirring conditions

2. Above dispersion was homogenized and then Nanomilled

3. Nanomilled drug slurry was adsorbed by spraying on lactose monohydrate, microcrystalline cellulose and crospovidone mixture in a fluidized bed granulator.

4. Granules obtained were sized and lubricated.

5. Lubricated granules were finally compressed into tablets

6. The tablets obtained were seal coated and then enteric coated.

### Example 6

| Sr. No. | Ingredients | Qty mg/tablet |
|---|---|---|
| | Binder Solution | |
| 1. | Rifaximin | 200.00 |
| 2. | Docusate Sodium IP | 2.00 |
| 3. | Hydroxypropylmethylcellulose 3 cps IP | 40.00 |
| 4. | Sodium lauryl sulphate IP | 5.50 |
| 5. | Sucrose IP | 69.00 |
| 6. | Purified water IP | q.s |
| | Dry Mix | |
| 7. | Lactose Monohydrate | 124.50 |
| 9. | Crospovidone IP | 73.00 |
| | Blending | |
| 10. | Microcrystalline cellulose (Avicel pH 102) | 50.00 |
| 11. | Sodium Starch Glycolate (Type A) | 20.00 |
| 12. | Talc | 3.00 |
| 13. | Croscarmellose sodium | 100.00 |
| | Lubrication | |
| 14. | Magnesium Stearate | 3.00 |
| | Total | 690.00 |
| | Enteric Coating | |
| 15. | Eudragit L 100-55 | 27.5 |
| 16. | Talc | 10.46 |
| 17. | Tri-Ethyl Citrate | 5.13 |
| 18. | Red Iron Oxide | 0.91 |
| 18. | Water | q.s. |
| 19. | Isopropyl Alcohol | q.s. |
| | Total | 734.00 |

Process:

1. Dispersion of rifaximin with Docusate sodium, HPMC, sodium lauryl sulphate and sucrose was prepared in purified water under stirring conditions

2. Above dispersion was homogenized and then Nanomilled

3. Nanomilled drug slurry was adsorbed by spraying on lactose monohydrate, and crospovidone mixture in a fluidized bed granulator.

4. Granules obtained were blended, sized and lubricated.

5. Lubricated granules were finally compressed into tablets

6. The tablets obtained were then enteric coated.

### Example 7

Dissolution of a composition according to the invention and a composition according to the prior art.

According to present invention dissolution study was carried out in an aqueous medium containing a surfactant 0.5% SLS. The paddle method (US Pharmacopoeia) was used under the following conditions: volume of medium 900 ml; medium temperature: 37° C.; blade rotation speed 50 rpm; samples taken: every 10 minutes.

TABLE 1

% dissolved

| Interval | Nanosized Rifaximin | Prior art |
|---|---|---|
| (mins) | tablets 200 mg | tablets 200 mg |
| 5 | 9 | 29.5 |
| 10 | 54 | 42 |
| 20 | 95 | 57 |
| 30 | 101 | 66.8 |

The composition according to present invention consisted of Rifaximin 200 mg tablets prepared according to Example 6. The prior art composition contained Rifaximin [200 mg] colloidal silicon dioxide, disodium edetate, glycerol palmitostearate, hypromellose, microcrystalline cellulose, propylene glycol, red iron oxide, sodium starch glycolate, talc, and titanium dioxide.

The results obtained are shown graphically in FIG. 1, on which the percentage of dissolution is shown. As shown in table 1 and FIG. 1, approximately 54% of the active from nano composition dissolved in 10 minutes and almost 100% of active dissolved within 30 minutes while prior art formulation dissolved only 66% in 30 minutes. These results clearly show that the compositions of the present invention have a dissolution profile which is distinctly better than the prior art composition.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “a propellant” includes a single propellant as well as two or more different propellants; reference to a “cosolvent” refers to a single cosolvent or to combinations of two or more cosolvents, and the like.

1. A composition comprising rifaximin in the form of particles, wherein substantially all the particles have a particle size less than or equal to 2 micrometres.

2. A composition according to claim 1, wherein substantially all the particles have a particle size less than or equal to 1 micrometre.

3. A composition according to claim 1, further comprising at least one surface stabilizer, at least one viscosity building agent and/or at least one polymer, wherein substantially all the particles have a particle size less than or equal to 2 micrometre.

4. A composition according to claim 3, wherein substantially all the particles have a particle size less than or equal to 1 micrometre.

5. A composition according to claim 3, wherein the surface stabilizer is a surfactant.

6. A composition according to claim 5, wherein the surfactant is an amphoteric, non-ionic, cationic or anionic surfactant.

7. A composition according to claim 5, wherein the surfactant is a polysorbates; sodium dodecyl sulfate (sodium lauryl sulfate); lauryl dimethyl amine oxide; docusate sodium; cetyl trimethyl ammonium bromide (CTAB); a polyethoxylated alcohol; a polyoxyethylene sorbitan; Octoxynol; N,N-dimethyldodecylamine-N-oxide; hexadecyltrimethylammonium bromide, polyoxyl 10 lauryl ether, brij, a bile salt, such as sodium deoxycholate or sodium cholate; a polyoxyl castor oil; nonylphenol ethoxylate; a Cyclodextrin; lecithin; methylbenzethonium chloride; a carboxylate; a sulphonate; a petroleum sulphonate; an alkylbenzenesulphonates; a naphthalenesulphonate; and olefin sulphonate; a sulphate surfactant; an alkyl sulphate; a sulphated natural oil or fat; a sulphated ester; a sulphated alkanolamide; an alkylphenol, optionally ethoxylated and sulphated; an ethoxylated aliphatic alcohol; polyoxyethylene; a carboxylic ester; a polyethylene glycol esters; an anhydrosorbitol ester or an ethoxylated derivative thereof; a glycol ester of a fatty acid; a carboxylic amide; a monoalkanolamine condensate; a polyoxyethylene fatty acid amide; a quaternary ammonium salt; an amine with amide linkages; a polyoxyethylene alkyl amine; a polyoxyethylene alicyclic amine; a N,N,N,N tetrakis substituted ethylenediamine; a 2-alkyl-1-hydroxyethyl-2-imidazoline; N-coco-3-aminopropionic acid or a sodium salt thereof; N-tallow-3-iminodipropionate disodium salt; N-carboxymethyl-n-dimethyl-n-9 octadecenyl ammonium hydroxide; n-cocoamidethyl-n-hydroxyethylglycine sodium salt; or mixtures thereof.

8. A composition according to claim 5, wherein the surfactant is docusate sodium and/or sodium lauryl sulphate.

9. A composition according claim 3, wherein the viscosity building agent is lactose; sucrose; saccharose; a hydrolyzed starch, such as maltodextrin; or a mixture thereof.

10. A composition according to claim 9, wherein the viscosity building agent is sucrose.

11. A composition according to claim 3, wherein the polymer is hydroxypropylcellulose; hydroxymethylcellulose; hydroxypropylmethylcellulose; a methylcellulose polymer; hydroxyethylcellulose; sodium carboxymethylcellulose; carboxymethylene hydroxyethylcellulose and/or carboxymethyl hydroxyethylcellulose; an acrylic polymer, such as acrylic acid, acrylamide, and maleic anhydride polymers and copolymers; or a blend thereof; or a mixture thereof.

12. A composition according to claim 12, wherein the polymer is hydroxypropylmethylcellulose.

13. A composition according to claim 1, wherein substantially all the particles have a particle size above 1 nanometre.

14. A composition according to claim 1, further comprising a pharmaceutically acceptable carrier, wherein said particles have been adsorbed onto the surface of the carrier.

15. A pharmaceutical composition comprising a composition according to claim 1.

16. A pharmaceutical composition comprising a composition according to claim 1, in combination with a pharmaceutically acceptable carrier.

17. A pharmaceutical composition according to claim 16, wherein the carrier comprises: one or more diluents or fillers; one or more binders; one or more lubricants; one or more glidants; one or more disintegrants; one or more preservatives; one or more humectants; one or more solution retarders; one or more absorption accelerators; one or more wetting agents; one or more adsorbents; one or more buffering agents; or a mixture thereof.

18. A pharmaceutical composition according to claim 16, which is for oral, ocular, parenteral, intracisternal, intravaginal, intraperitoneal, or buccal administration.

19. A pharmaceutical composition according to claim 16, which is for oral administration.

20. A pharmaceutical composition according to claim 17, which is a solid oral dosage form.

21. A pharmaceutical composition according to claim 19, which is in the form of a capsule, tablet, powder, or granules.

22. A pharmaceutical composition according to claim 20, further comprising an enteric coating.

23. A composition according to claim 1 for use in treating a bacterial infection.

24. A method of using a composition according to claim 1, comprising formulating a medicament comprising a compound of claim 1 for treating a bacterial infection.

25. A method of treating a bacterial infection comprising administering a therapeutically effective amount of a composition according to claim 1 to a patient in need thereof.

26. A process for preparing a pharmaceutical composition, which process comprises the steps of: homogenizing rifaximin, at least one surface stabiliser, at least one viscosity building agent, and at least one polymer to produce a homogenized dispersion of the rifaximin in the surface active agent, the viscosity building agent and the polymer; milling said homogenized dispersion to produce a slurry of particles having a particle size less than or equal to 2 micrometres; and adsorbing the milled slurry on carrier to form granules.

27. A composition according to claim 2, further comprising at least one surface stabilizer, at least one viscosity building agent and/or at least one polymer, wherein substantially all the particles have a particle size less than or equal to 2 micrometre.

28. A composition according to claim 4, wherein the surface stabilizer is a surfactant.

29. A composition according to claim 6, wherein the surfactant is a polysorbates; sodium dodecyl sulfate (sodium lauryl sulfate); lauryl dimethyl amine oxide; docusate sodium; cetyl trimethyl ammonium bromide (CTAB); a polyethoxylated alcohol; a polyoxyethylene sorbitan; Octoxynol; N,N-dimethyldodecylamine-N-oxide; hexadecyltrimethylammonium bromide, polyoxyl 10 lauryl ether, brij, a bile salt, such as sodium deoxycholate or sodium cholate; a polyoxyl castor oil; nonylphenol ethoxylate; a Cyclodextrin; lecithin; methylbenzethonium chloride; a carboxylate; a sulphonate; a petroleum sulphonate; an alkylbenzenesulphonates; a naphthalenesulphonate; and olefin sulphonate; a sulphate surfactant; an alkyl sulphate; a sulphated natural oil or fat; a sulphated ester; a sulphated alkanolamide; an alkylphenol, optionally ethoxylated and sulphated; an ethoxylated aliphatic alcohol; polyoxyethylene; a carboxylic ester; a polyethylene glycol esters; an anhydrosorbitol ester or an ethoxylated derivative thereof; a glycol ester of a fatty acid; a carboxylic amide; a monoalkanolamine condensate; a polyoxyethylene fatty acid amide; a quaternary ammonium salt; an amine with amide linkages; a polyoxyethylene alkyl amine; a polyoxyethylene alicyclic amine; a N,N,N,N tetrakis substituted ethylenediamine; a 2-alkyl-1-hydroxyethyl-2-imidazoline; N-coco-3-aminopropionic acid or a sodium salt thereof; N-tallow-3-iminodipropionate disodium salt; N-carboxymethyl-n-dimethyl-n-9 octadecenyl ammonium hydroxide; n-cocoamidethyl-n-hydroxyethylglycine sodium salt; or mixtures thereof.

30. A composition according to claim 6, wherein the surfactant is docusate sodium and/or sodium lauryl sulphate.

31. A composition according to claim 7, wherein the surfactant is docusate sodium and/or sodium lauryl sulphate.

32. A composition according claim 4, wherein the viscosity building agent is lactose; sucrose; saccharose; a hydrolyzed starch, such as maltodextrin; or a mixture thereof.

33. A pharmaceutical composition according to claim 17, which is for oral, ocular, parenteral, intracisternal, intravaginal, intraperitoneal, or buccal administration.

34. A pharmaceutical composition according to claim 20, which is in the form of a capsule, tablet, powder, or granules.

35. A pharmaceutical composition according to claim 21, further comprising an enteric coating.
